(19) United States
(12) Patent Application Publication
(10) Pub. No.: US 2002/0090630 A1
(43) Pub. Date: Jul. 11, 2002

(54) Method of sequence determination for nucleic acid

(75) Inventor: Makoto Hazama, Hyogo (JP)

Correspondence Address: ARMSTRONG,WESTERMAN & HATTORI, LLP, 1725 K STREET, NW., SUITE 1000, WASHINGTON, DC, 20006, US

(73) Assignee: Shimadzu Corporation, Kyoto (JP)

(21) Appl. No.: 09/991,631

(22) Filed: Nov. 26, 2001

(30) Foreign Application Priority Data
Nov. 29, 2000 (JP) 2000-362648

(51) Int. Cl. C12Q 1/68; G06F 19/00; G01N 33/48; G01N 33/50

(52) U.S. Cl. 435/6; 702/20

(57) ABSTRACT

Peaks are extracted in a certain range of starting points of signals from migration waveforms. The peaks are classified on the basis of signal strengths for obtaining signal strength ratios of four groups classified. Corresponding bases are allocated to the four groups classified for obtaining a matrix value from the signal strength ratios of peak waveforms of the respective base groups. Base sequence is determined with the matrix value. Thus, the matrix value can be obtained from actual sample migration without employing an exclusive reagent kit.

### BACKGROUND OF THE INVENTION

1. Field of the Invention

The present invention relates to a method of determining base sequence of nucleic acid such as DNA (deoxyribonucleic acid).

2. Description of the Prior Art

The base sequence of DNA is determined on the basis of strengths (heights) of signal peaks obtained in four types of detection parts which selectively detect four types of wavelengths respectively when DNA fragment specimens labeled with fluorochromes varying with bases are electrophoresed.

FIG. 2 (quoted from “ABIPRISM (registered trademark of Applied Biosystems) BigDye (registered trademark of Applied Biosystems) Terminator Cycle Sequencing Ready Reaction Kit”) shows standardized emission spectra of dRhodamin in a fluorochrome terminator. Four types of detection parts are set to most sensitively detect four types of fluorochromes (dR110, dR6G, dTAMRA and dROX) respectively.

However, the emission spectra of the fluorochromes are definitely not sharp and the foot portions thereof evidently leak into the right and left detection parts. For example, all of the detection parts for the four types of fluorochromes detect the peak waveform of the base A (adenine) labeled with dR6G with differences between the strengths, as shown in FIG. 3. The current signal strength ratios (Pa:Pt:Pg:Pc) are constant, and hence it follows that the peak waveform of only the base A is exclusively obtained through inverse transformation based on this value. This also applies to the remaining three types of fluorochromes.

The signal strengths in the detection parts are expressed as follows:

Signal Strength Ratio of Peak Waveform of Base A=APa(=1):Apg:Apc:APt

Signal Strength Ratio of Peak Waveform of Base G=GPa:Gpg(=1):Gpc:GPt

Signal Strength Ratio of Peak Waveform of Base C=CPa:Cpg:Cpc(=1):CPt

Signal Strength Ratio of Peak Waveform of Base T=TPa:Tpg:Tpc:TPt(=1)

Emission Strength by Base A=Ia

Emission Strength by Base G=Ig

Emission Strength by Base C=Ic

Emission Strength by Base T=It

Signal Strength detected in Detection Part Da for Base A=Oa

Signal Strength detected in Detection Part Dg for Base G=Og

Signal Strength detected in Detection Part Dc for Base C=Oc

Signal Strength detected in Detection Part Dt for Base T=Ot

At this time, the relation between the emission strengths (Ia, Ig, Ic and It) by the fluorochromes and the signal strengths (Oa, Og, Oc and Ot) received is expressed in the following matrix:

$$( \begin{matrix}Oa \\ Og \\ Oc \\ Ot\end{matrix} ) = ( \begin{matrix}APa & GPa & CPa & TPa \\ APg & GPg & CPg & TPg \\ APc & GPc & CPc & TPc \\ APt & GPt & CPt & TPt\end{matrix} ) ( \begin{matrix}Ia \\ Ig \\ Ic \\ It\end{matrix} )$$

$$\underline{↗} matrix M$$

Therefore, both sides of the above expression may be multiplied by the inverse matrix of the matrix M, in order to obtain original signals, i.e., the signal waveforms (Ia, Ig, Ic and It) of the bases (fluorochromes) from the signal waveforms (Oa, Og, Oc and Ot) obtained. This inverse matrix is the matrix value.

Also when a peak signal overlaps with that of another base, the waveform detected is conceivably mere addition of spectra.

Therefore, when signal strength ratios as to the four types of fluorochromes are obtained, the peak waveforms of the respective fluorochromes (four types of bases) are obtained by expressing the same in a matrix and multiplying the original detected peak waveforms by the inverse matrix thereof. It is to correctly obtain the signal strength ratios, to obtain the matrix value of the fluorochromes.

In general, in order to obtain the matrix value of fluorochromes, bases labeled with fluorochromes varying with bases are migrated one by one for measuring the strengths (heights) of signal peaks obtained in four types of detection parts selectively detecting four types of wavelengths respectively.

The matrix value of fluorochromes is somewhat specific depending on the fluorochromes labeling the respective bases and a signal detection system including an optical system, and hence it is necessary to set a new value every time when hardware for migration/detection is adjusted or components are exchanged. On the other hand, once the value is set, no re-setting is required unless inconvenience takes place, that is, the matrix value is displaced, for some reason.

Four types of fluorochromes are mixed in a reagent kit for fluorochrome terminator labeling from the first. Therefore, a specific reagent kit containing fluorochromes independently of each other is necessary for matrix value calibration.

Furthermore, it is necessary to make migration for calibration with this exclusive reagent kit. This is made only at the start and hence barely results in a problem when migration is routinely made. When an experiment of changing conditions of a migration is carried out, an evaluation experiment of the reagent kit itself is carried out or adjustment of the optical system is repeated. However, it is extremely troublesome and costly.

When the exclusive reagent kit is employed, the bases must be labeled with different fluorochromes and migrated one by one, since firstly, it is impossible to explicitly distinguish the bases to which the obtained peak waveforms belong. Also, when employing a method of assuming a detection part having the highest signal strength for peak waveforms as that for the base (for example, the base A in the case of FIG. 3), there is no guarantee that the peak waveform consists of only one base and absolutely doesn't overlap with the remaining bases due to the difference in mobility between the bases. Assuming that the mobility of guanine G, for example, is larger than that of adenine A as schematically shown in FIG. 4, it is possible that peaks A and G partially overlap with each other. When the peaks partially overlap with each other, the signal strength ratios are changed and a correct matrix value cannot be obtained.

### SUMMARY OF THE INVENTION

In order to solve the aforementioned problem, a method capable of extracting a peak waveform exclusively consisting of only one base per base can be found out.

In order to implement this, an object of the present invention is to provide a method of obtaining a matrix value from actual sample migration without employing an exclusive reagent kit.

The present invention provides a method of performing matrix transformation on a waveform signal obtained from a detection part for each fluorochrome by fluorochrome terminator labeling employing a plurality of fluorochromes having different fluorescent waveforms for obtaining a signal waveform per base, and determining the base sequence of nucleic acid on the basis thereof, wherein the method obtains a matrix value for performing the matrix transformation from actual sample migration through steps of:

\mathring{1} extracting peaks from a proper range;

\mathring{2} eliminating peaks having irregular peak intervals;

\mathring{3} classifying the peaks into four groups corresponding to the types of bases;

\mathring{4} obtaining signal strength ratios of the classified four groups;

\mathring{5} allocating the corresponding bases to the classified four groups; and

\mathring{6} obtaining the matrix value by signal strength ratios of peak waveforms of the respective base groups.

According to the present invention, those accomplishing prescribed conditions are extracted from peaks obtained from actual sample migration and the matrix value is obtained with these peaks, whereby the base sequence can be determined without employing an exclusive reagent kit.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a flow chart showing one aspect of the present invention;

FIG. 2 illustrates standardized emission spectra of dRhodamin;

FIG. 3 illustrates signal peak waveforms of the base A detected in respective detection parts;

FIG. 4 schematically shows shifting of peak positions resulting from mobility values varying with bases; and

FIG. 5 illustrates inverted signal strengths.

### DESCRIPTION OF THE PREFERRED EMBODIMENTS

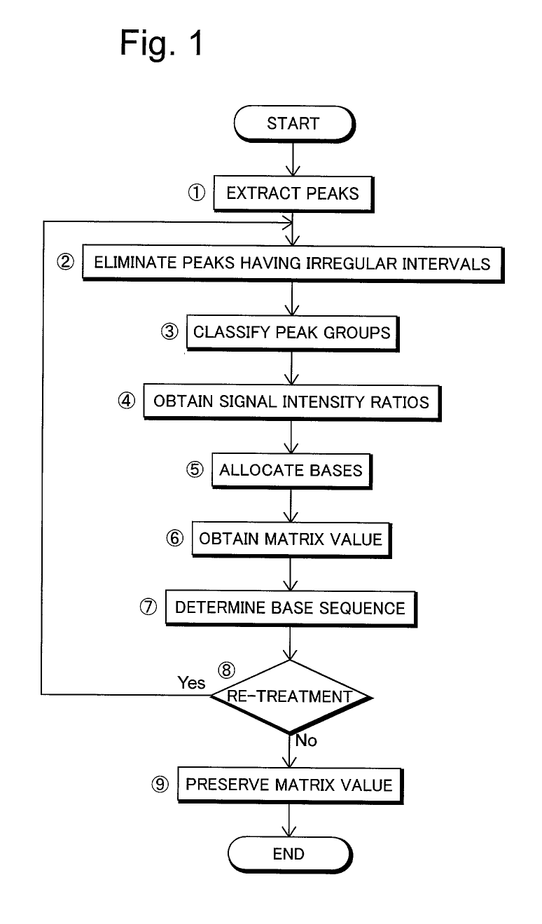

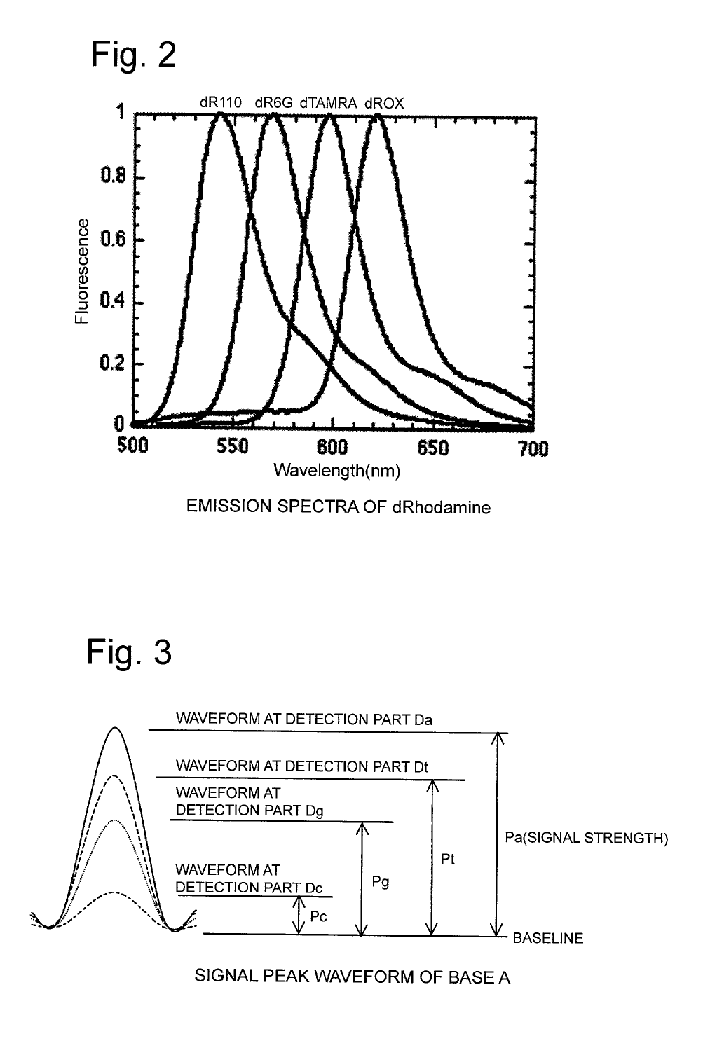

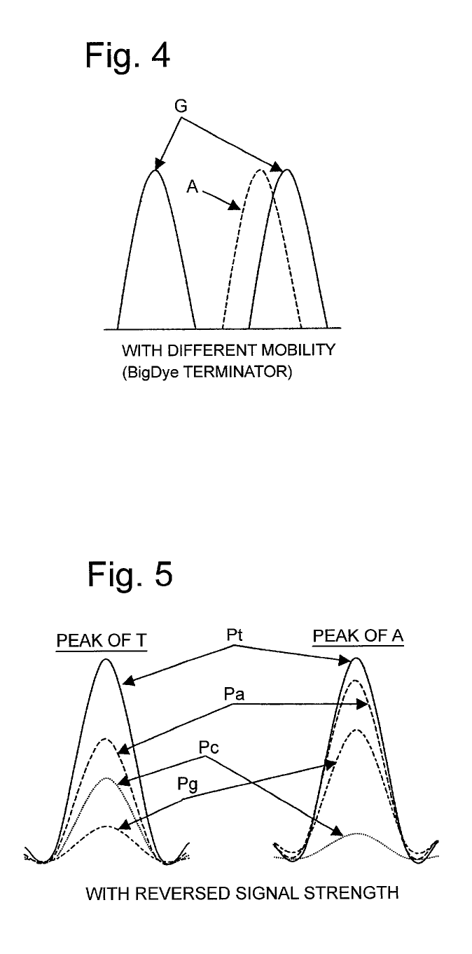

A method according to a first embodiment of the present invention shall now be described with reference to FIG. 1.

\mathring{1} To extract peaks

As to migration waveforms, clear peak waveforms having excellent signal-to-noise ratios are generally obtained in starting portions of signals. Therefore, the operation is commenced from extraction of peaks in a certain range of the starting points of the signals. In this case, the reference of the peaks is that the strength of the largest fluorochrome signal is larger than the minimum level for peak detection in a used base caller (program for base sequencing). This is because the signal-to-noise ratio deteriorates in a small signal.

\mathring{2} To eliminate peaks having irregular intervals

Peaks generally overlap with each other in base sequence serially including bases having small and large mobility values, and peak intervals are irregular in front and back portions in this case. When detecting such portions and eliminating the peaks in the portions, most parts of a problem regarding to mobility can be solved.

\mathring{3} To classify peaks in response to signal strengths

For example, an A (adenine) group [Pa>Pt>Pg>Pc], a T (thymine) group [Pt>Pa>Pc>Pg], a G (guanine) group [Pg>Pa>Pt>Pc] and a C (cytosine) group [Pc>Pt>Pa>Pg] in the BigDye terminator. While four bases must originally be classified into four types, there is a possibility that additionally classified peak groups appear due to Sanger's reaction, a failure of purification or a problem of noise. In this case, classification of upper four groups having larger peak numbers is selected on the premise that such abnormal peaks have a small appearance frequency. Also, when the signal strengths of fluorochromes of separated wavelengths are larger than those of fluorochromes of adjacent wavelengths, peaks thereof are eliminated as abnormality. Overlapping peaks resulting from difference in mobility uneliminable in \mathring{2} can be further eliminated by this treatment.

\mathring{4} To obtain signal strength ratios of the classified four groups

The signal strength ratios are calculated and obtained per group. Various calculation methods such as mean values or central values can be utilized.

\mathring{5} To allocate the corresponding bases to the classified four groups

It is essential that Pa has the strongest signal strength ratio in a peak of A (adenine) and Pt has the strongest signal strength ratio in a peak of T (thymine). However, the signal strengths may be reversed due to sensitivity setting of detectors or the like. For example, a peak of A (adenine) may exhibit [Pt≧Pa] to appear T (thymine) due to inferior sensitivity of the detector for adenine or superior sensitivity of the detector for thymine. When A (adenine) exhibits [Pt≧Pa>Pg>Pc] and T (thymine) exhibits [Pt≧Pa>Pc>Pg] as shown in FIG. 5, however, it is distinguishable by a third largest signal and recognized as A (adenine) from an adjacent wavelength of Pg.

When both exhibit [Pt≧Pa>Pg>Pc], strength ratios Pg/Pa of adjacent wavelengths of Pg are compared as to two groups for assuming the group exhibiting the larger value as A (adenine).

\mathring{6} To obtain a matrix value from signal strength ratios of peak wavelengths of the respective base groups

Signal strength ratios of peak wavelengths of the respective groups to which the bases are allocated are obtained for creating a matrix of the signal strength ratios. An inverse matrix of the matrix is calculated for obtaining a matrix value.

\mathring{7} To perform ordinary base calling (base sequencing)

Matrix transformation on waveform signals is performed with the obtained matrix value for obtaining signal waveforms of the bases and determining the base sequence on the basis thereof.

\mathring{8} To obtain a further optimum matrix value from the result of base calling

A base caller generally performs reliability-oriented weighting on sequenced bases. In this step, bases (peak signals) weighted as almost reliably correct are extracted as to the overall data range, and the steps \mathring{2} to \mathring{4} are carried out again with waveform signal information thereof. The currently treated peak groups are generally superior as signal waveforms to the peak groups obtained in the step \mathring{1}, and have larger numbers of peaks since the data range is not only over the starting points of the signals but also over a wide range. Therefore, a correct matrix value having higher precision is obtained.

\mathring{9} To preserve the obtained matrix value

It follows that base calling is performed with this matrix value from the succeeding time. When an index varying with migration conditions and a reagent kit is added to the matrix value, distribution of the matrix value is simplified by calling the same.

Needless to say, it may not be possible to create the matrix value due to a failure of Sanger's reaction or purification, trouble of a polymer or a gel or a problem of noise as to target (original) sample migration. For example, no clear difference is obtained in the numbers of peaks included in the groups between the upper four groups to be classified and remaining groups in the step \mathring{3}, or the number of bases (peaks) weighted as correct is small in the step \mathring{8}. Particularly when a large number of peaks to be relied upon cannot be obtained in the step \mathring{8}, there is a large possibility that the original matrix value is erroneous. Of course such sample migration must not be employed as the target sample migration for obtaining the matrix value.

While a method carried out without limiting various conditions has been described in the above, a simple method limiting various conditions shall now be described as a second embodiment of the present invention.

The conditions to be limited are the following two points

(1) The sensitivities of detection parts are so set that Pa is the strongest for a peak of A (adenine), Pt is the strongest for T (thymine), Pg is the strongest for G (guanine) and Pc is the strongest for C (cytosine). This adjustment generally results in such a secondary effect that the signal strengths of the respective bases are uniformed as a result. This is extremely preferable for a base caller, and hence the strengths may paradoxically be slightly reversed so that the peak heights of the bases are uniformed.

(2) The difference in mobility or strength between fluorochromes, recognized by a reaction reagent kit, is previously embedded in an algorithm.

The item (1) shows basic contents required for adjusting a migration system in order to ensure a signal-to-noise ratio and perform precise migration. Also in the item (2), no reaction reagent kits having absolutely different characteristics are employed per migration, but it is general to create/tune the base caller while taking an existing reaction reagent kit into consideration, resulting in improving precision of the base caller. In other words, both the items (1) and (2) are ordinary measures for performing accurate base sequencing in a DNA sequencing system rather than conditions to be limited and do not lead to being a significant burden.

The tendencies of the order and ratios of signal strengths detected in the detection parts for the respective fluorochromes can be though approximate, conclusively predicted to a certain extent when the sensitivities are adjusted in the item (1) and the difference in strength between the fluorochromes is recognized in the item (2). Therefore, the items (1) and (2) are sufficient to successively decide extracted peaks from the first and classifying the same into four types of bases. Consequently, the contents of the treatments in the steps \mathring{3} and \mathring{5} can be notably reduced.

When the mobility levels of the fluorochromes are recognized from the item (2), dispersion of peak intervals can be readily predicted for improving accuracy of peaks to be sorted out in peak selection of the step \mathring{2}. For example, G (guanine) exhibits the highest mobility in a BigDye terminator, and hence there is such a tendency that peak intervals are extremely narrowed in front of G (guanine) as compared with the back side unless peaks in front of and at the back of G (guanine) are identically those of G (guanine). This is not abnormality of the peak intervals but is a normal state. In this case, this peak signal of G (guanine) is effective unless the peak interval on the front side is so narrow that the same influences the signal strength ratios.

The second embodiment shall be described in further detail.

The reaction reagent kit is an ET terminator (registered trademark of amersham pharmacia biotech). In the ET terminator, only T (thymine) has slightly slow mobility while the remaining three bases may be regarded as being substantially identical in mobility to each other. Emission wavelengths of fluorochromes are in order of G (guanine)<T (thymine)<A (adenine)<C (cytosine) from a shorter wavelength side. As to adjustment of sensitivities of the detection parts, priority is given to uniformity of peak strengths of the respective bases while allowing slight reversal of the strengths.

The procedure shall now be described.

[1] Extraction of Peaks

Four types of bases (signal peaks) are extracted within a range of about 50 bp (base pair) from starting points of signals.

[Peak Extraction of G (guanine)]

Peaks larger than A (adenine) and C (cytosine) and larger than 90% of the strength of T (thymine) are extracted as peak candidates for G (guanine).

[Peak Extraction of T (thymine)]

Peaks larger than 90% of the strengths of A (adenine) and G (guanine) are extracted as peak candidates for T (thymine).

[Peak Extraction of A (adenine)]

Peaks larger than G (guanine) and larger than 90% of the strengths of T (thymine) and C (cytosine) are extracted as peak candidates for A (adenine).

[Peak Extraction of C (cytosine)]

Peaks larger than G (guanine), A (adenine) and T (thymine) are extracted as peak candidates for C (cytosine).

[2] Calibration of Peak Interval

Intervals in front of and at the back of the extracted peaks are confirmed, and then two peaks having narrow intervals, and peaks having wide front and back intervals are removed from the candidates. Considering that T (thymine) has low mobility, displacement of about half peak intervals is allowed in front of and at the back of T (thymine). When at least three peaks of the same base are continuous, peak signals excluding both ends are preferentially left.

[3] To calculate signal strength ratios and then obtain a matrix value

Signal strength ratios of the peaks left in calibration are calculated for obtaining central values for the respective bases as representative values. Central values are employed instead of mean values since mean values sometimes exhibit values displaced from true values in a system having large noise.

A matrix is created from the four types of representative values, and an inverse matrix thereof is obtained as a matrix value.

[4] To perform matrix transformation on signal waveforms for carrying out base calling.

[5] To obtain a more optimum matrix value from the result of base calling.

Bases (peak signals) weighted as almost reliably correct as the result of base calling are extracted as to the overall data range, and new matrix value is calculated with the signal strength ratios thereof.

[6] To preserve the matrix value in a file

The matrix value is preserved in a file with addition of the recognition number of a DNA sequencing unit employed for this migration and the mark of the ET terminator. When the ET terminator is thereafter employed for migration in this unit, it follows that the base caller automatically refers to this matrix value.

As in the embodiment, tuning of methodology corresponding to the system remarkably depends on a migration system including the reaction reagent kit and the detection parts. Sometimes the procedure may be out of order, or absolutely reversed condition settings may be required.

However, a proper measure responsive to the circumstances is necessary, and is further the condition for a high-speed base caller having high precision.

Although the present invention has been described and illustrated in detail, it is clearly understood that the same is by way of illustration and example only and is not to be taken by way of limitation, the spirit and scope of the present invention being limited only by the terms of the appended claims.

What is claimed is:

1. A method of sequence determination for nucleic acid performing matrix transformation on a waveform signal obtained from a detection part. for each fluorochrome by fluorochrome terminator labeling employing a plurality of fluorochromes having different fluorescent waveforms for obtaining a signal waveform every base, and determining a base sequence on the basis thereof, wherein the method obtains a matrix value for performing the matrix transformation from actual sample migration through steps of:
\mathring{1} extracting peaks from a proper range;
\mathring{2} eliminating peaks having irregular peak intervals;
\mathring{3} classifying the peaks into four groups corresponding to the types of bases;
\mathring{4} obtaining signal strength ratios of classified the four groups;
\mathring{5} allocating the corresponding bases to the classified four groups; and
\mathring{6} obtaining the matrix value by signal strength ratios of peak waveforms of the respective base groups.

2. The method of sequence determination according to claim 1, wherein
the proper range in the step \mathring{1} is a certain range of starting points of signals.

3. The method of sequence determination according to claim 1, wherein
the peaks extracted in the step \mathring{1} are such peaks that the strength of the maximum fluorochrome signal is larger than the minimum standard for peak detection in a used sequence determination program.

4. The method of sequence determination according to claim 1, wherein
such peaks that signal strengths of fluorochromes of separate waveforms are larger than signal strengths of fluorochromes of adjacent waveforms are eliminated in the step \mathring{1}.

5. The method of sequence determination according to claim 1, wherein
the four groups classified in the step \mathring{3} are upper four groups having large peak numbers.

6. The method of sequence determination according to claim 1, wherein
the signal strength ratios in the step \mathring{4} are either mean values or central values.

7. The method of sequence determination according to claim 6, wherein
the signal strength ratios are central values.

8. The method of sequence determination according to claim 1, wherein
the bases are allocated in the step \mathring{5} by, when the types of maximum detection signals of four groups are different each other, allocating the types of these maximum detection signals as the base species of respective the groups.

9. The method of sequence determination according to claim 1, wherein
the bases are allocated in the step \mathring{5} on the basis of, when the types of maximum detection signals of two groups are identical to each other, the types of the third largest detection signals of the groups.

10. The method of sequence determination according to claim 1, wherein
the base sequence is determined with obtained the matrix value for thereafter obtaining a matrix value again with peak signals of the determined base sequence.

11. The method of sequence determination according to claim 1, wherein
conditions are limited thereby simplifying treatment in at least one of the steps \mathring{1} to \mathring{6}.
